(19) United States
(12) Patent Application Publication
(10) Pub. No.: US 2011/0097268 A1
(43) Pub. Date: Apr. 28, 2011

(54) RADIOHALOIMATINIBS AND METHODS OF THEIR SYNTHESIS AND USE IN PET IMAGING OF CANCERS

(75) Inventors: William Bornmann, Missouri City, TX (US); Mian Alauddin, Houston, TX (US); Juri Gelovani, Missouri City, TX (US); Pradip Ghosh, Houston, TX (US); Liwei Guo, Birmingham, AL (US); Dongmei Han, Houston, TX (US); David Maxwell, Pearland, TX (US); Uday Mukhopadhyay, Houston, TX (US); Zhenghong Peng, Missouri City, TX (US); Aleksandr Shavrin, Porter, TX (US); Yunming Ying, Houston, TX (US)

(73) Assignee: BOARD OF REGENTS, THE UNIVERSITY OF TEXAS SYSTEM

(21) Appl. No.: 12/310,333

(22) PCT Filed: Aug. 22, 2007
(86) PCT No.: PCT/US07/76501

§ 371 Date: Dec. 9, 2010

Related U.S. Application Data
(60) Provisional application No. 60/823,324, filed on Aug. 23, 2006.

(51) Int. Cl. A61K 51/04; C07D 403/14; A61P 35/00

(52) U.S. Cl. 424/1.89; 544/295

(57) ABSTRACT

We disclose methods of synthesizing radiohalidated organic compounds and their use in positron emission tomography (PET) imaging of cancer cells.

### BACKGROUND OF THE INVENTION

The present invention relates generally to the field of radiolabeled markers for positron emission tomography (PET) imaging. More particularly, it concerns methods of synthesizing various radiolabeled markers and methods for their use.

A large number of kinases are known, in both humans and other animals. Kinases transfer phosphate groups to specific substrates. Human kinases have been grouped into several categories: tyrosine kinases, which phosphorylate tyrosine residues of proteins and of which KIT and ABL are members; serine-threonine kinases, which phosphorylate serine or threonine residues of proteins; TK (thymidine kinases); STE; CK (creatine kinases); CMGC; AGC; CAMK; and others. Protein kinases are capable of phosphorylating a large percentage of the total proteins in an animal proteome. As a result, evolution has led to the diligent regulation of the activity of kinases. Failure of proper regulation of kinase activity is a frequent cause of cancer.

Activating mutations of the activation loop of KIT are associated with certain human neoplasms, including the majority of patients with systemic mast cell disorders, as well as cases of seminoma, acute myelogenous leukemia (AML), and gastrointestinal stromal tumors (GISTs) and hypopigmentary disorders. Mast cell is a hematopoietic lineage dependent on Kit signaling for growth, differentiation, and survival. Mast cells are found in excessive numbers in tissues in a heterogeneous group of disorders collectively known as mastocytosis. Systemic mastocytosis has been found to be associated with activating codon 816 mutations of the c-kit gene. The mutation was used as a tracking marker to elucidate the clonal nature of mastocytosis. Improved knowledge of the mechanisms causing pathological mast cell growth will lead to the discovery of novel treatment options including drugs targeting the mutated Kit protein. Kit-D816 mutations are associated with impaired event-free and overall survival. Activating mutations of receptor tyrosine kinases are associated with distinct genetic subtypes in AML. The KIT-D816 mutations confer a poor prognosis to AML1-ETO-positive AML and should therefore be included in the diagnostic workup. Constitutive KIT tyrosine kinase activity was hypothesized to provide growth and survival signals to GIST. Small-molecule tyrosine kinase inhibitor imatinib mesylate is a potent inhibitor of wild-type (WT) KIT and certain mutant KIT isoforms and has become the standard of care for treating patients with metastatic GIST. However, Distinct forms of tyrosine kinase domain (TKD), juxtamembrane domain, exon 8, and internal tandem duplication (ITD) mutations of c-KIT, were observed in about 46% of core binding factor leukemia (CBFL) patients. Activation loop mutations of c-Kit involving codon D816 that are typically found in AML, systemic mastocytosis, and seminoma are insensitive to imatinib mesylate (IC50>5-10 micromol/L), and acquired KIT activation loop mutations can be associated with imatinib mesylate resistance in GIST. Imatinib binding and c-KIT inhibition is abrogated by the c-Kit kinase domain I missense mutation Va1654Ala. Dasatinib (formerly BMS-354825) is a small-molecule, ATP-competitive inhibitor of SRC and ABL tyrosine kinases with potency in the low nanomolar range. Some small-molecule SRC/ABL inhibitors also have potency against WT KIT kinase. Dasatinib might inhibit the kinase activity of both WT and mutant KIT isoforms.

The role of ¹⁸F-FDG in the staging and early prediction of response to therapy of recurrent gastrointestinal stromal tumors by Imatinib mesylate (Gleevec® or STI571) has been extensively studied by Gayed et al. who has demonstrated ¹⁸F-FDG is superior to CT in predicting early response to therapy. In addition, more recently, Jager et al. [2] as well as other investigators have confirmed this observation. Noteworthy is the observation that GIST has been shown to share immunohistochemical, ultrastructural and histogenic similarities with the interstitial cells of Cajal. Both GIST and the interstitial cells of Cajal express c-Kit, the receptor tyrosine kinase that is the protein product of the c-kit proto-oncogene. C-Kit is universally phosphorylated in GISTs. It has also been found that c-Kit from GIST cells have demonstrated a high frequency of mutations that lead to constitutive activation of the c-Kit tyrosine kinase in the absence of stimulation by its physiologic ligand (stem cell factor) which in turn causes uncontrolled stimulation of downstream signaling cascades with aberrant cellular proliferation and resistance to apoptosis[3]. While these studies are of the great clinical value, they cannot identify patients who will be responsive to Imatinib mesylate chemotherapy prior to treatment.

### SUMMARY OF THE INVENTION

One embodiment of the present invention relates to a method of synthesizing a radiohaloimatinib or a salt thereof, comprising condensing a radiohalopiperazine benzoic acid chloride with N-(2-Methyl-5-aminophenyl)-4-(3-pyridyl)-2-pyrimidine-amine, to yield the radiohaloimatinib or a salt thereof.

One embodiment of the present invention relates to a method of positron emission tomography (PET) imaging of cancer cells in a mammal to detect the levels of expression or activity of a kinase by the cancer cells, comprising administering to the mammal a composition containing a radiohaloimatinib or a salt thereof, and imaging the mammal with PET.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1B. Synthesis scheme for haloSTI.

FIG. 2A-2B. Synthesis scheme for haloSTI.

FIG. 3. HPLC for products of Synthesis scheme for haloSTI.

FIG. 4. Simulation of docking of STI analogs with kinase.

FIG. 5: Radio-synthetic scheme for ¹⁸F-STI.

FIG. 6: Purification of ¹⁸F-STI: Sem-prep. Column:

50% MeCN/(0.1% HCOONH₄ in H₂O); Flow: 4 ml/min.

FIG. 7: HPLC chromatogram of ¹⁸F-STI: Co-injected with standard F-STI:

Analytical Column: 50% MeCN/(0.1% HCOONH₄ in H₂O); Flow: 1 ml/min.

FIG. 8A-8B: Transformed data for transforms of STI-F2, STI-OH, and STI-571.

### DESCRIPTION OF ILLUSTRATIVE EMBODIMENTS

“Radiohalide,” as used herein, refers to any halogen isotope which decays by emitting a positron. Examples of radiohalides include, but are not limited to, ¹⁸F, ¹²⁴I, ¹²⁵I, and ¹³¹I.

One embodiment of the present invention relates to a method of synthesizing a radiohaloimatinib or a salt thereof, comprising condensing a radiohalopiperazine benzoic acid chloride with N-(2-Methyl-5-aminophenyl)-4-(3-pyridyl)-2-pyrimidine-amine, to yield the radiohaloimatinib or a salt thereof.

Imatinib has the following general structure:

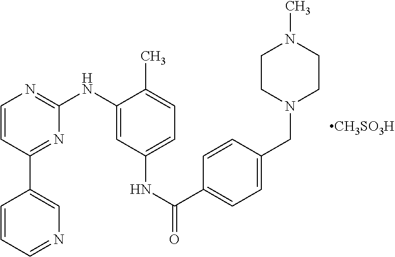

In one embodiment, the reagents for the synthesis can be prepared beginning with treatment of a 2-alkyl-5-nitroaniline with acid in ethanol followed by the addition of cyanoamide to give the corresponding 2-alkyl-5-nitroaniline-guanidine nitrate or other salt depending on the chosen acid. 3-acetyl-pyridine can be treated with alkyl dialkoxyforamide to give 3-dialkylamino-1-3-pyridyl-2-alkene-1-one. The 2-alkyl-5-nitroaniline-guanidine salt can be treated with 3-dialkylamino-1-3-pyridyl-2-alkene-1-one and base in refluxing organic solvent, such as isopropanol, to give N-(2-alkyl-5-nitroaryl)-4-(3-pyridyl))-2-pyrimidine-amine, which can be subsequently hydrogenated, such as by 10% palladium on carbon, to give N-(2-alkyl-5-aminoaryl)-4-(3-pyridyl)-2-pyrimidine-amine. Haloimatinib synthesis can consist of the reaction of α-halo-p-toluoylic acid with a substituted piperazine in ethanol followed by treatment with concentrated halide acid (e.g., HCl) to give the corresponding benzoic acid which is subsequently treated with thionyl halide to give the corresponding acid halide dihydrohalide. Subsequent condensation with N-(2-alkyl-5-aminoaryl)-4-(3-pyridyl)-2-pyrimidine-amine in pyridine affords the haloimatinib.

Radiohalidation of the haloimatinib can be performed as will be described in more detail below.

In PET, a short-lived radioactive tracer isotope, such as ¹⁸F (half life≈110 min), which decays by emitting a positron, is chemically incorporated into a metabolically active molecule and injected into a living subject. Injection into blood circulation is the most common, but PET is not limited thereto. After injection, the metabolically active molecule becomes concentrated in tissues of interest that contain molecules, enzymes, or other structures which interact with the metabolically active molecule and the subject is placed in the imaging scanner. Decay of the short-lived isotope emits a positron. After traveling a short distance (typically no more than a few millimeters) the positron annihilates with an electron, producing a pair of annihilation photons (similar to gamma rays) moving in opposite directions. These are detected when they reach a scintillator material in the scanning device, creating a burst of light which is detected by photomultiplier tubes.

The most significant fraction of electron-positron decays result in two 511 keV photons being emitted at almost 180 degrees to each other, allowing localization of their source along a straight line of coincidence. Using statistics collected from tens-of-thousands of coincidence events, a set of simultaneous equations for the activity of each parcel of tissue along many lines of coincidence can be solved, and thus a map of locations and radioactivities in the body may be plotted. The resulting map shows the tissues in which the molecular probe has become concentrated, and can be interpreted by nuclear medicine physician or radiologist in the context of the patient's diagnosis or treatment plan.

One embodiment of the present invention relates to molecular probes analogous to anti-cancer drugs which target given proteins expressed by given alleles of various oncogenes, which probes can be used in PET to determine whether the given proteins are targeted by the molecular probe and hence whether the anti-cancer drug would be likely to be efficacious against the cancer characterized by activity of the given allele of the particular oncogene.

The short half-lives of many radionuclides useful in PET make necessary the rapid incorporation of the radionuclides into metabolically active molecules.

One embodiment of the present invention relates to a method of positron emission tomography (PET) imaging of cancer cells in a mammal to detect the levels of expression or activity of a kinase by the cancer cells, comprising administering to the mammal a composition containing a radiohaloimatinib or a salt thereof, and imaging the mammal with PET.

In one embodiment, the radiohaloimatinib is selected from the group consisting of

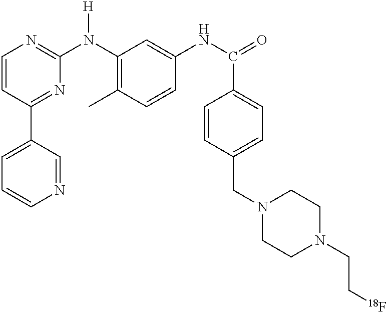

and salts thereof.

In one embodiment, the cancer cells are selected from the group consisting of seminoma cells, acute myelogenous leukemia (AML) cells, and gastrointestinal stromal tumor (GIST) cells.

Though not to be bound by theory, it is our hypothesis that PET imaging with a radiohaloimatinib or a salt thereof, such as [¹²⁴I] or [¹⁸F] of a Gleevec analog, could help to identify GIST tumor patients with high c-Kit expression/activity, as having higher radiohaloimatinib uptake and retention levels, and who would respond favorably to therapy with c-Kit inhibitor Gleevec, both in terms of an early decrease in radiohaloimatinib uptake and retention, as well as by a gradual regression in tumor size. Similarly, and again not to be bound by theory, it is our hypothesis that PET imaging with a radiohaloimatinib or a salt thereof, such as [¹²⁴I] or [¹⁸F] Gleevec analog, may help to identify GIST tumor patients with low c-Kit expression/activity, as those having a low radiohaloimatinib uptake and retention, and would respond poorly to therapy with c-Kit specific inhibitor Gleevec.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of STI analogs

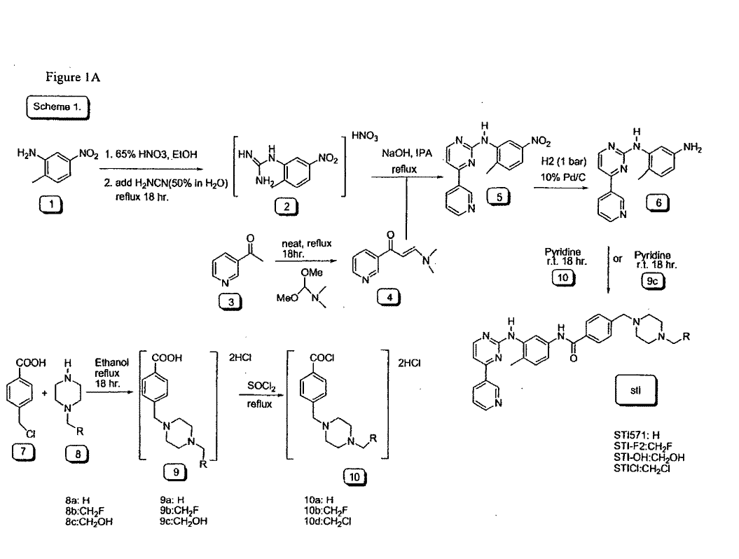

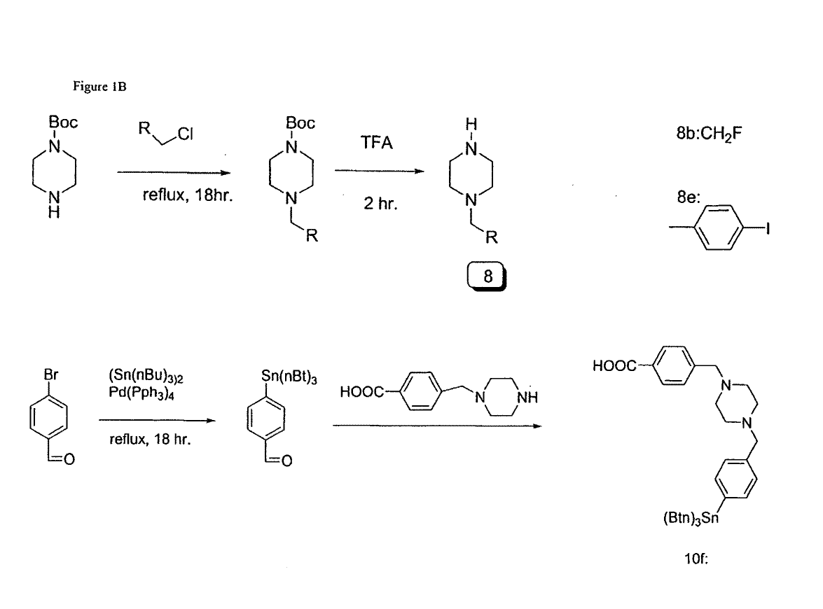

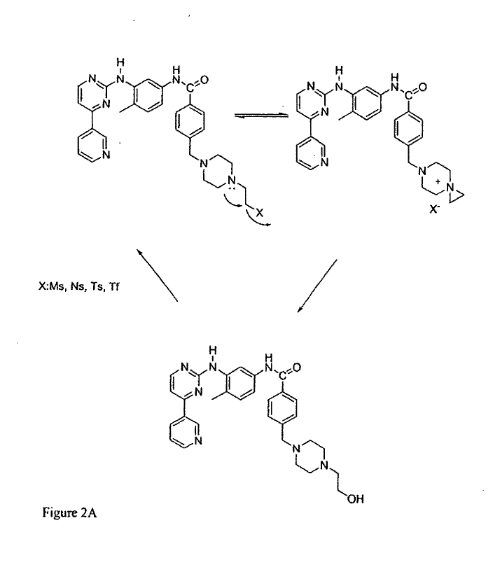

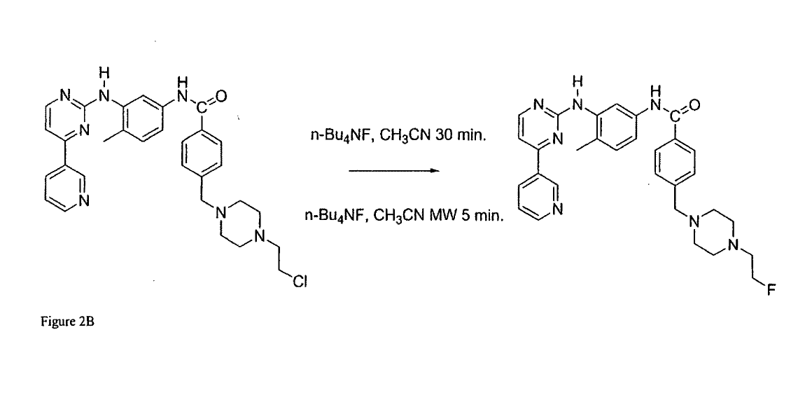

Turning to FIG. 1, the synthesis begins with treatment of 2-methyl-5-nitroaniline (1) with 65% nitric acid in ethanol followed by the addition of cyanoamide to give the corresponding 2-methyl-5-nitroaniline-guanidine nitrate (2). 3-Acetyl-pyridine was treated with methyl dimethoxyforamide to give 3-dimethylamino-1-3-pyridyl-2-propene-1-one (4). The nitrate salt (2) is treated with (4) and sodium hydroxide in refluxing isopropanol to give N-(2-Methyl-5-nitrophenyl)-4-(3-pyridyl))-2-pyrimidine-amine (5) which is subsequently hydrogenated with 10% palladium on carbon to give N-(2-Methyl-5-aminophenyl)-4-(3-pyridyl)-2-pyrimidine-amine (6). The gleevec analog synthesis will consist of the reaction of α-chloro-p-toluoylic acid (8) with a substituted piperazine in ethanol followed by treatment with con. HCl to give the corresponding benzoic acid (9) which is subsequently treated with thionyl chloride to give the corresponding acid chloride dihydrochloride (10). Subsequent condensation with N-(2-Methyl-5-aminophenyl)-4-(3-pyridyl)-2-pyrimidine-amine (6) in pyridine affords the STI analogs.

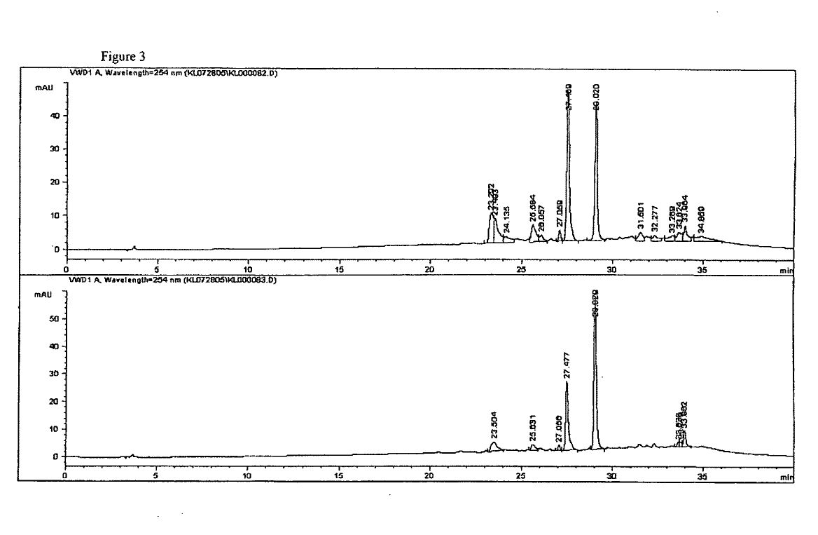

Attempts to make radiolabeling precursor, triflate, mesylate active leaving group from STI-OH failed because it was easily degraded into a mixture of starting material alcohol and STI-Cl during the purification of chromatography on Silica Gel. The behavior of these β-triflate amines may be due to the participation effect of nitrogen which cause triflate to leave by aliphatic chain cyclisation giving the aziridinium ion, which in turn reacts with the nucleophilic agent OH⁻, Cl⁻. In order to get a pure precursor, STI-Cl analog was selected as a target compound. The hydroxyl-Benzoic acid (9c) was treated with thionyl chloride to achieve the corresponding chloro-acid chloride dihydrochloride (10d). Subsequent condensation with N-(2-Methyl-5-aminophenyl)-4-(3-pyridyl)-2-pyrimidine-amine (6) in pyridine affords the STI-Cl. STI-Cl was not stable and decomposed at room temperature over two weeks. The Sti-Cl was treated with tetra ammonia fluoride at 80° C. in acetonitrile for 30 mins to generate cold Sti-F. Sti-Cl was also treated with tetra ammonia fluoride at 80° C. in acetonitrile microwave for 5 mins to generate cold Sti-F. The traditional heating method gave more yield and the microwave condition gave more clean reaction. (FIG. 3).

### Example 2

### Docking of STI-571 and F/I-Labeled Analogs

Protein Structure and Ligand Preparation

The crystal structures of STI-571 bound to c-Kit (PDB code 1T46) and Abl (PDB code 1IEP) were obtained from the Protein Data Bank.[1] The structures were brought into Sybyl 7.2[2] and all additional chains, waters, and ions were removed before docking. The STI-571 ligand from 1IEP was extracted and modified with correct atom types for Tripos force field and protonated at the piperazine nitrogen furthest from the phenyl group. A minimization was completed using the Powell method and gradient cutoff of 0.05 kcal/(mol Å). This structure was the basis for creating the STI-571_F2 and STI-571_I_{—}3, and these structures were also minimized in the same manner.

Docking with FlexX

Docking was originally completed with the v1.13.2L and v1.20.1 of FlexX [3, 4] as distributed in Sybyl. The primary docking region was selected as all residues within 6.5 Å of the STI-571. Ten configurations were requested for each ligand and formal charge assignment for the ligand was template based.

Results

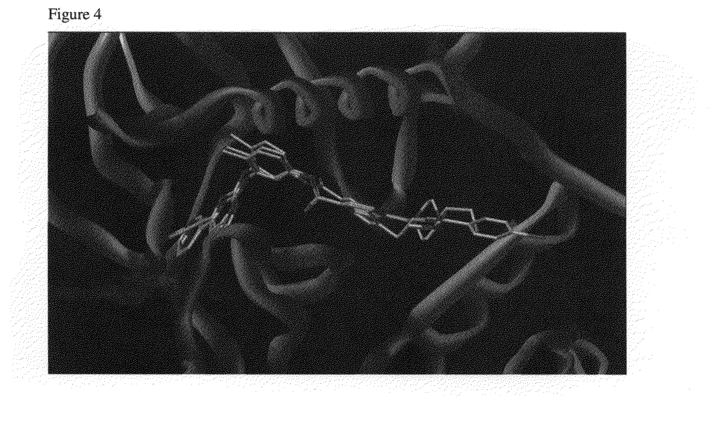

The following table represents the FlexX docking results for the highest ranked configuration of each ligand/receptor combination. The highest ranked configuration for STI-571 aligns with the crystal structure with a heavy atom RMSD of 0.58, suggesting the algorithm is capable of correctly docking this and similar ligands. The compounds had similar scores and nearly identical binding configurations for the highest ranking configuration. There is no evidence to suggest that these modifications would cause any unfavorable steric overlap. See FIG. 4. Therefore, we would expect that the modified molecules should retain similar activity in comparison to STI-571 for c-Kit and abl kinase.

| Ligand | c-Kit | ABL |
|---|---|---|
| STI-571 | −41.469 | −37.091 |
| STI-571_I_3 | −37.695 | −40.580 |
| STI-571_F2 | −38.544 | −38.386 |

### Example 3

A new gleevec analogue (¹⁸F-STI) was prepared by the reaction of STI-Cl with tetrabutylammonium[¹⁸F]fluoride. The crude product was purified by HPLC to obtain the desired pure product (¹⁸F-STI). The radiochemical yield was 8-16% with an average of 12% (d.c.) in 12 runs and radiochemical purity was >99% with specific activity >74 GBq/mmol at the end of synthesis. The synthesis time was 65-75 min from the end of bombardment (EOB).

Results and discussion

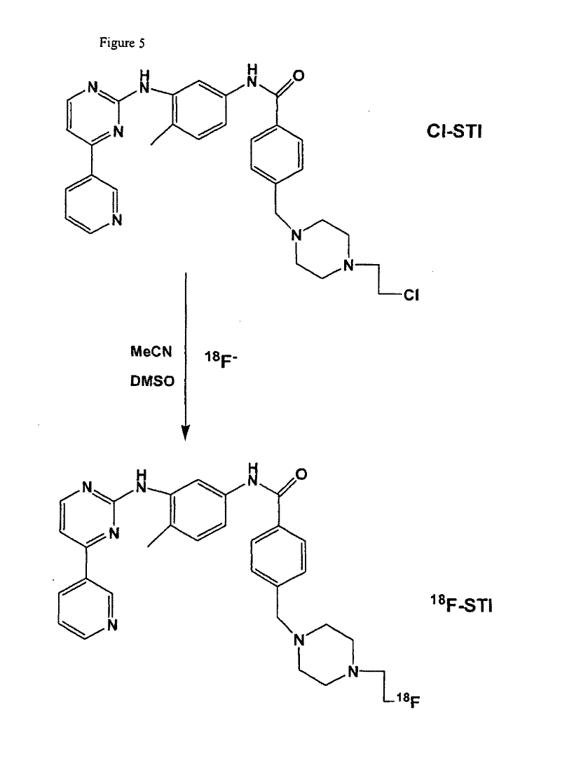

FIG. 5 represents the scheme for synthesis of ¹⁸F-STI analogue. Non-radiolabel compound F-STI was prepared by the reaction of Cl-STI with n-Bu₄NF in dry acetonitrile and DMSO (1:3) at 90° C. for 25 min. The chemical yield was 43% after chromatographic purification. This fluorocompound was characterized by ¹⁹F NMR spectroscopy in addition to ¹H NMR, and mass spectrometry. ¹H NMR spectrum of F-STI showed a peak (dt) at 4.58 ppm with J=47.4 Hz, a typical geminal coupling constant between fluorine and hydrogen, and J=4.8 Hz for H—H coupling. ¹⁹F NMR spectrum (decoupled) of F-STI showed single peaks at −218.23 ppm. The coupled spectra of this compound showed multiplets due to the long range coupling between fluorine and hydrogen in addition to the geminal coupling.

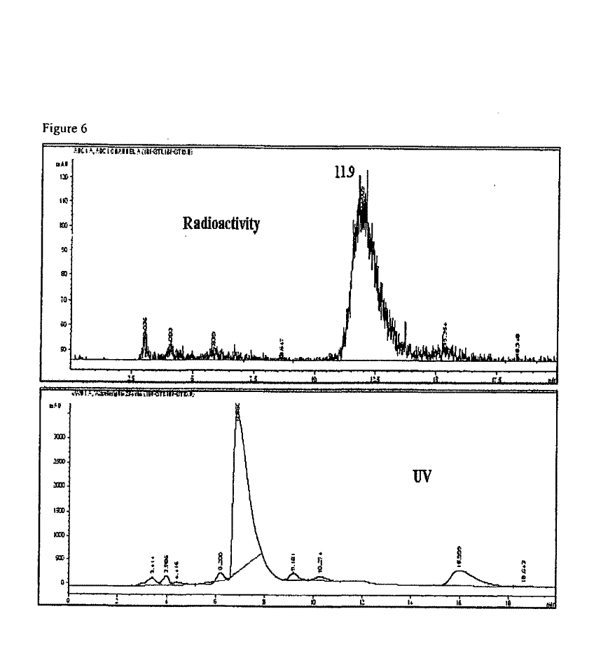

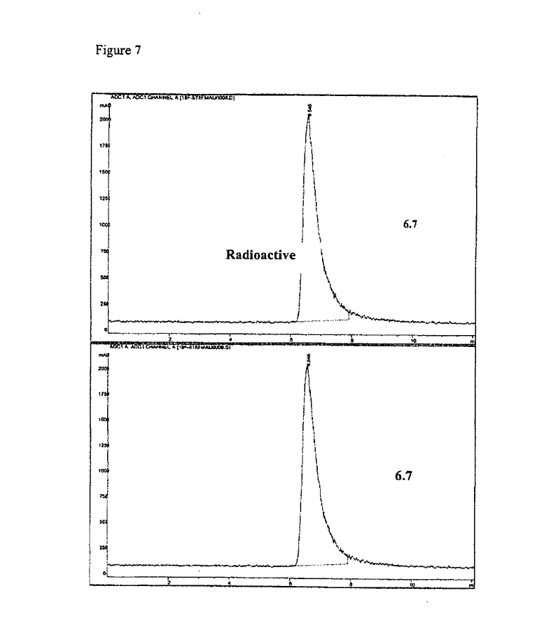

Radiolabeled compound ¹⁸F-STI was prepared by fluorination of the Cl-STI precursor with n-Bu₄N¹⁸F followed by HPLC purification. n-Bu₄N¹⁸F was prepared in situ from n-Bu₄NHCO₃ and aqueous H¹⁸F using 0.40 ml (1% solution, ˜7 mmol) of n-Bu₄NHCO₃ to elute the activity from the ion exchange cartridge.^{1,2} Following radiofluorination of the precursor the crude reaction mixture was passed through a silica-gel cartridge (900 mg, Alltech), and the crude product was eluted with 30% methanol in dichloromethane. Silica gel holds the unreacted [¹⁸F]-fluoride from the product and helps to avoid overloading of the HPLC column with high levels of free fluoride during purification. The recovered ¹⁸F-labeled STI was purified on a semi preparative HPLC and was eluted at 11.6 using a 50% MeCN and 50% water containing 0.1% HCOONH₄ solvent system with a flow of 4 ml/min (FIG. 6). Used 0.1% ammonium formate in water to improve the peak shape. Quality control analysis of the final product was performed on analytical HPLC using the same solvent system at a flow of 1 ml/min. A co-injection of the final product with an authentic sample of F-STI showed that the radiolabeled compound and standard F-STI was co-eluted at 6.7 min (FIG. 7). The radiochemical yield of this synthesis was 8-16%, with an average 12% in 12 runs (Corrected for decay). The radiochemical purity was >99% with specific activity >74 GBq/mmol. The synthesis time was 65-75 min from the end of bombardment (EOB).

Reagents and Instrumentation

All reagents and solvents were purchased from Aldrich Chemical Co. (Milwaukee, Wis.), and used without further purification. Solid phase extraction cartridges (silica gel, 900 mg) were purchased from Alltech Associates (Deerfield, Ill.). Thin layer chromatography (TLC) was performed on pre-coated Kieselgel 60 F254 (Merck) glass plates.

Proton and ¹⁹F NMR spectra were recorded on a Brucker 300 MHz spectrometer using tetramethylsilane as an internal reference and hexafluorobenzene as an external reference, respectively, at The University of Texas MD Anderson Cancer Center.

High performance liquid chromatography (HPLC) was performed on a 1100 series pump (Agilent, Germany), with built in UV detector operated at 254 nm, and a radioactivity detector with single-channel analyzer (Bioscan, Washington D.C.) using a semi-preparative C₁₈ reverse phase column (Alltech, Econosil, 10×250 mm, Deerfield, Ill.) and an analytical C₁₈ column (Rainin, Microsorb-MV, 4.6×250 mm, Emeryville, Calif.). A 50% acetonitrile and 50% water containing 0.1% ammonium formate (MeCN/aqueous NH₄-formate) solvent system was used for purification of the radiolabeled product. Same solvent system was used for quality control analysis of F-STI on analytical HPLC.

### Preparation of F-STI

Compound Cl-STI (30 mg, 0.06 mmol) was dissolved in dry MeCN (3 mL) and DMSO (1 mL) in a sealed v-vial under argon. To the above solution, n-Bu₄NF (1M, 40 μL) was added and the mixture was heated at 90° C. for 25 min in a heating block. The reaction mixture was cooled to room temperature and evaporated under vacuum, and the residue was dissolved in CH₂Cl₂ (30 mL). The solution was washed with H₂O (3×30 mL). The organic phase was dried (MgSO₄), evaporated to dryness and purified on a silica gel column using 60% acetone in hexane. The pure compound F-STI (12.5 mg) was obtained in 43% yield.

¹H NMR (CDCl₃) δ: 9.25 (1H), 8.70 (1H), 8.59 (1H), 8.53 (2H), 7.85 (3H), 7.42 (3H), 7.27 (1H), 7.22 (2H), 7.04 (1H), 4.58 (dt, 2H, J_{HF}=47.4 Hz & J_{H-H}=4.8 Hz, CH₂F), 2.78 (1H), 2.69 (1H), 2.35 (s, 3H, CH₃). ¹⁹F NMR (δ): −218.23 (s, decoupled), −217.97 to −218.50 (m, coupled).

MS: M+1, 526.5

### Preparation of [¹⁸F]-STI

The aqueous [¹⁸F]fluoride was trapped in anion exchange cartridge (ABX, Germany) and eluted with a solution of n-Bu₄NHCO₃ (400 μL, 1% by wt.) into a v-vial and the solution evaporated azeotropically with acetonitrile (1.0 mL) to dryness at 79-80° C. under a stream of argon. To the dried n-Bu₄N¹⁸F, a solution of Cl-STI (5-6 mg) in anhydrous acetonitrile (0.45 mL) and DMSO (0.15 ml) at the ratio of 3:1 was added and the mixture was heated at 90° C. for 25 min. The reaction mixture was cooled, passed through a silica gel cartridge (Alltech), and eluted with 30% methanol in dichloromethane (2 ml). After evaporation of the solvent under a stream of argon at 80° C., the crude mixture was diluted with HPLC solvent (1.2 mL) and purified by HPLC. The desired product was isolated and radioactivity was measured in a dose calibrator (Capintec, Ramsey, N.J.). Solvent was evaporated and the product was redissolved in saline. The final product was analyzed onto an analytical column and co-injected with an authentic standard compound to confirm its identity.

Biology

The final product was assayed for kinase binding using techniques known in the art.

Stock:

F1, F2, OH—10 mM in H2O

I 3, NO2—10 mM in DMSO

571—1 mM in DMSO

YY—100 uM in DMSO

Drug dilution:

F1, F2, OH, I 3, NO2, 571—(1/stock+9/H2O)

YY—(2/stock+1/H2O)×5

All drugs (exc. YY) contain CH₃SO₃H.

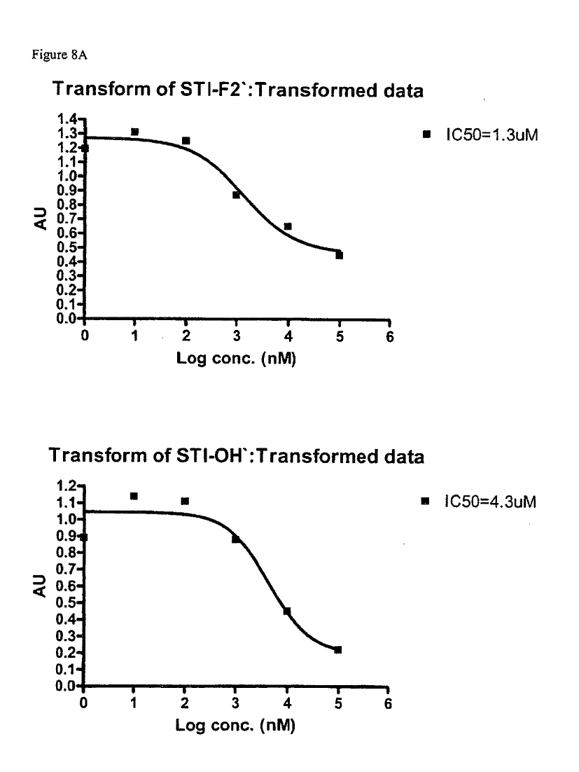

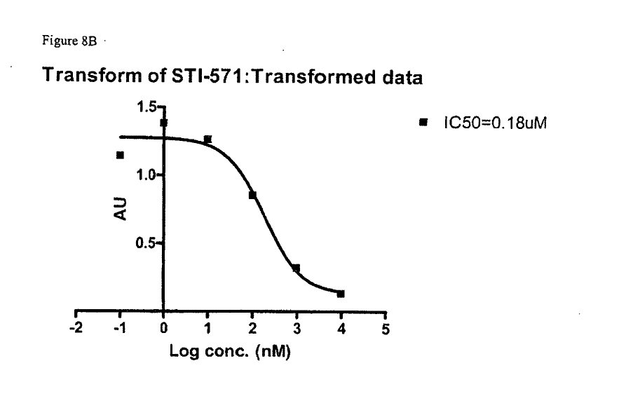

Results are shown in FIG. 8.

### Example 4

### Radiosynthesis Uptake and Distribution

Material and Methods:

All chemicals and solvents were obtained from Sigma-Aldrich (Milwaukee, Wis.) of Fisher Scientific (Pittsburgh, Pa.) and used without further purification. Analytical HPLC was performed on a Varian Prostar system, with a Varian Microsorb-MW C18 column (250×4.6 mm; 5μ) using the following solvent system A=H₂O/0.1% TFA and B=acetonitrile/0.1% TFA. Varian Prepstar preparative system equipped with a Prep Microsorb-MWC18 column (250×41.4 mm; 6μ; 60 Å) was used for preparative HPLC with the same solvent systems. Mass spectra (ionspray, a variation of electrospray) were acquired on an Applied Biosystems Q-trap 2000 LC-MS-MS. UV was measured on Perkin Elmer Lambda 25 UV/Vis spectrometer. IR was measured on Perkin Elmer Spectra One FT-IR spectrometer. ¹H-NMR and ¹³C-NMR spectra were recorded on a Brucker Biospin spectrometer with a B-ACS 60 autosampler. (600.13 MHz for ¹H-NMR and 150.92 MHz for ¹³C-NMR), Chemical shifts (δ) are determined relative to d4-methanol (referenced to 3.34 ppm (δ) for ¹H-NMR and 49.86 ppm for ¹³C-NMR). Proton-proton coupling constants (J) are given in Hertz and spectral splitting patterns are designated as singlet (s), doublet (d), triplet (t), quadruplet (q), multiplet or overlapped (m), and broad (br). Flash chromatography was performed using Merk silica gel 60 (mesh size 230-400 ASTM) or using an Isco (Lincon, Nebr.) combiFlash Companion or SQ16x flash chromatography system with RediSep columns (normal phase silica gel (mesh size 230-400ASTM) and Fisher Optima™ grade solvents. Thin-layer chromatography (TLC) was performed on E. Merk (Darmstadt, Germany) silica gel F-254 aluminum-backed plates with visualization under UV (254 nm) and by staining with potassium permanganate or ceric ammonium molybdate.

### 2-methyl-5-nitrophenyl-guanidine nitrate(2)³

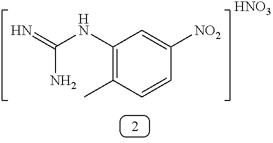

### 2-Methyl-5-nitroaniline (100 g, 0.657 mol) was dissolved in ethanol (250 ml), and 65% aqueous nitric acid solution (48 ml, 0.65 mol) was added thereto. When the exothermic reaction was stopped, cyanamide (41.4 g) dissolved in water (41.4 g) was added thereto. The brown mixture was reacted under reflux for 24 hours. The reaction mixture was cooled to 0° C., filtered, and washed with ethanol:diethyl ether (1:1, v/v) to give 2-methyl-5-nitrophenyl-guanidine nitrate (98 g). R_{f}=0.1 (Methylene chloride:Methanol: 25% Aqueous ammonia=150:10:1). MS: 195.2 (M+H); ¹H-NMR (DMSO-d₆)=1.43 (s, 3H), 6.59 (s, 3H), 6.72-6.76 (d, 1H), 7.21-7.27 (m, 1H), 8.63-8.64 (br, 1H).

### 3-dimethylamino-1-(3-(6-Methyl-pyridyl))-2-propen-1-one³

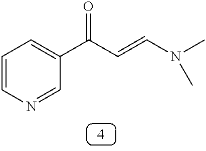

3.Acetylpyridine (100 g, 0.19 mol) was added to dimethylformamide dimethylacetal (156.5 g, 1.27 mol), and the mixture was reacted under reflux for 23 hours. After the reaction mixture was cooled to 0° C., a mixture of diethyl ether and hexane (3:2, v/v) (500 ml) was added and the whole mixture was stirred for 4 hours. The resulting solid was filtered and washed with a mixture of diethyl ether and hexane (500 ml, 3/2, v/v) to give 3-dimethylamino-1-(3-pyridyl)-2-propen-1-one (120 g, 85%). R_{f}=0.46 (Methylene chloride:Methanol=9:1).

NMR in agreement with the reference.

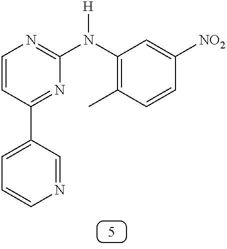

Dimethylamino-1-(3-pyridyl)-2-propen-1-one (25 g, 0.14 mol), 2-methyl-5-nitrophenyl-guanidine nitrate (36 g, 0.14 mol), and sodium hydroxide powder (6.5 g, 0.163 mol) were dissolved in isopropanol and reacted under reflux for 18 hours. The reaction solution was cooled to 0° C., filtered, washed with isopropanol and methanol, and dried to give N-(2-methyl-5-nitrophenyl)-4-(3-pyridyl)-2-pyrimidine-amine (20 g).

R^{f}=0.6 (Methylene chloride:Methanol=9:1). NMR in agreement with the reference.

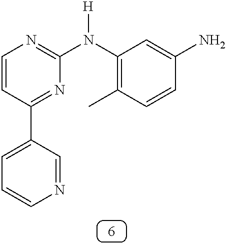

N-(2-methyl-5-nitrophenyl)-4-(3-pyridyl)-2-pyrimidine-amine (35 g, 0.114 mol) and stannous chloride dihydrate (128.5 g, 0.569 mol) were dissolved in a solvent mixture of ethyl acetate and ethanol (250 ml, 10/1, v/v), and the reaction solution was refluxed for 4 hours. The solution was cooled to room temperature, washed with 10% aqueous sodium hydroxide solution, and concentrated to give N-(5-amino-2-methylphenyl)-4-(3-pyridyl)-2-pyrimidine-amine (35 g).

R_{f}=0.45 (Methylene chloride:Methanol=9:1). NMR in agreement with the reference.

### Preparation of 4-(4-methylpiperazinomethyl)benzoic acid dihydrochloride⁴

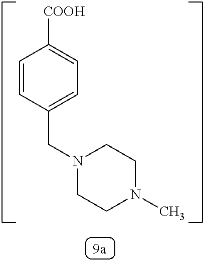

To a well-stirred suspension consisting of 17.1 g. (0.10 mole) of α-chloro-p-toluoylic acid in 150 ml. of absolute ethanol under a nitrogen atmosphere at room temperature (˜20° C.), a solution consisting of 44.1 g. (0.44 mole) of N-methylpiperazine dissolved in 50 ml. of ethanol was added dropwise. The resulting reaction mixture was refluxed for a period of 16 hours and then cooled to room temperature. The cooled reaction mixture was concentrated in vacuo and the thus obtained residue partitioned between 100 ml. of diethyl ether and 100 ml. of 3N aqueous sodium hydroxide. The separated aqueous layer was then washed three times with 100 ml. of diethyl ether, cooled in an ice-water bath and subsequently acidified with concentrated hydrochloric acid. The resulting solids were filtered and air-dried, followed by trituration with 150 ml. of boiling isopropyl alcohol and stirring for a period of two minutes. After filtering while hot and drying the product there were obtained 9.4 g. (35%) of pure 4-(6-methylpiperazinomethyl)benzoic acid dihydrochloride as the hemihydrate, m.p. 310°-312° C. MS: 235.1 (M+H); ¹H NMR (D₂O) δ 8.04 (d, J=8.21 Hz, 2H), 7.59 (d, J=8.21 Hz, 2H), 3.50 (s, 2H), 3.63 (br, 8H), 2.97 (s, 3H); ¹³C NMR δ 170.18, 133.13, 131.91, 130.90, 60.22, 50.61, 48.77, 43.25.

### Preparation of 4-(4-methylpiperazinomethyl)benzoyl chloride dihydrochloride⁴

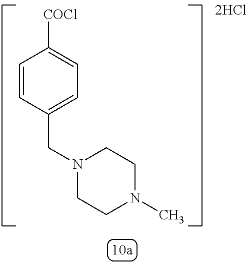

To 20 g. (0.065 mole) of 4-(4-methylpiperazinomethyl)benzoic acid dihydrochloride under a nitrogen atmosphere, there were added 119 ml. of thionyl chloride (194 g., 1.625 mole) to form a beige-white suspension. The reaction mixture was refluxed for 24 hours and then cooled to room temperature (˜20° C.). The resulting suspension was filtered, and the recovered solids were washed with diethyl ether and dried to ultimately afford 17.0 g. (81%) of pure 4-(4-methylpiperazinomethyl)benzoyl chloride dihydrochloride.

### Preparation of N-{5-[4-(4-methyl piperazine methyl)-benzoylamido]-2-methylphenyl}-4-[3-(4-methyl)-pyridyl]-2-pyrimidine amine (free base)

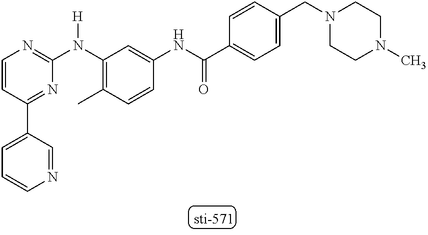

A mixture of N-(2-methyl-5-aminophenyl)-4-pyridyl))-2-pyrimidine-amine (7) 5 g (18 mmol) and 4-(4-methylpiperazinomethyl)benzoyl chloride dihydrochloride (10a) 5 g (20 mmol) were stirred in 50 ml anhydrous pyridine at 20° C. for 18 hours. The reaction mixture was concentrated in vacuum. The residue was subjected to silica gel chromatography using 5% Methanol (7M NH₃) in DCM. 5 g obtained. Yield 60%. MS: 494.5 (M+H).

### Preparation of N-{5-[4-(4-methyl piperazine methyl)-benzoylamido]-2-methylphenyl}-4-[3-(4-methyl)-pyridyl]-2-pyrimidine amine (mesylate salt)

The above obtained free base sti571 were dissolved in 15 ml of ethanol and added one equivalent of methyl sulfonic acid. The solution was reacted at 45° C. for 2 hours and the solvent was evaporated to give the mesylate salt.

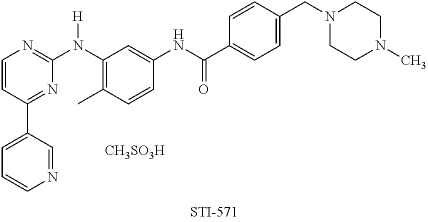

MS: 494.5 (M+H). NMR: ¹H NMR (D₂O) δ 8.63 (s, 1H), 8.20 (d, J=5 Hz, 1H), 8.04 (d, J=5 Hz, 1H), 7.98 (d, J=8.0 Hz, 1H), 7.83 (s, 1H), 7.49 (d, J=7.5 Hz, 2H), 7.26 (d, J=8.0 Hz, 2H), 7.05 (dd, 1H), 6.96 (d, J=8.0 Hz, 1H), 6.85 (m, 2H), 3.58 (s, 2H), 3.0 (br, 8H), 2.80 (s, 3H), 2.75 (s, 3H).

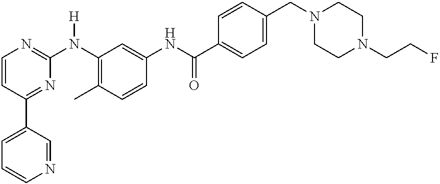

MS: 526.5 (M+H). NMR: ¹H NMR (MeOD) δ 10.17 (s, 1H), 9.28 (s, 1H), 8.98 (s, 1H), 8.69 (d, J=3 Hz, 1H), 8.51 (dd, J=4.8, 1.8 Hz, 1H), 8.48 (dd, J=7.8, 1.8 Hz, 1H), 8.10 (s, 1H), 7.91 (d, J=6.6, 2H), 7.52 (t, J=7.8 Hz, 1H), 7.49 (d, J=7.8 Hz, 1H), 7.431 (m, 3H), 7.21 (d, J=5.0 Hz, 1H), 4.52 (t, J=48.0 Hz H—F coupling, 4.9 Hz, 2H), 3.55 (s, 2H), 3.34 (b, 4H), 2.65 (d, J=48.0 Hz H—F coupling 2H), 2.50 (b, 4H), 2.23 (s, 3H). ¹³C NMR (MeOD) δ 165.71, 162.10, 161.68, 159.92, 151.84, 148.68, 138.28, 137.68, 134.88, 134.32, 132.71, 130.48, 129.13, 128.05, 124.23, 117.72, 117.24, 107.99, 82.72, 81.63, 61.98, 57.97, 57.85 (¹³C-¹⁹F 1-3 coupling 18 Hz), 53.32, 52.97 (¹³C-¹⁹F 1-2 coupling 52.82 Hz).: ¹⁹F NMR (MeOD) δ −217.20 (¹H decoupled), −217.05, −217.10, −217.14, −217.15, −217.19, −217.22, −217.27, −217.32

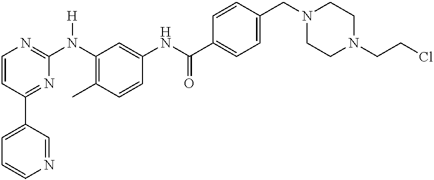

MS: 542.2 (M+H). NMR: ¹H NMR (MeOD) δ 9.28 (s, 1H), 8.68 (dd, 1H), 8.51 (dd, J=4.8, 1.8 Hz, 1H), 8.48 (dd, J=7.8, 1.8 Hz, 1H), 8.11 (s, 1H), 7.91 (d, J=6.6, 2H), 7.52 (t, J=7.8 Hz, 1H), 7.49 (d, J=7.8 Hz, 1H), 7.431 (m, 3H), 7.21 (d, J=5.0 Hz, 1H), 3.67 (t, J=8.0 Hz, 2H), 3.63 (s, 2H), 3.58 (s, 2H), 2.50 (br, 8H), 2.63 (t, J=8.0 Hz, 2H), 2.23 (s, 3H).

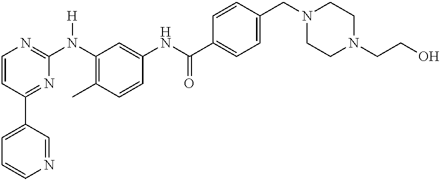

MS: 524.5 (M+H). NMR: ¹H NMR (MeOD) δ 9.29 (d, 1H), 8.65 (dd, J=6.5, 1.5 Hz, 1H), 8.60 (d, J=8.0 Hz, 1H), 8.48 (d, J=5 Hz, 1H), 8.22 (s, 1H), 7.93 (d, J=8.5 Hz, 2H), 7.56 (dd, J=8.0, 1.5 Hz, 1H), 7.51 (t, 3H), 7.43 (t, J=8.0 Hz, 2H), 7.37 (d, J=5.0 Hz, 1H), 7.28 (d, J=8.5 Hz, 1H), 3.71 (t, J=8.0 Hz, 2H), 3.63 (s, 2H), 3.58 (s, 2H), 2.70 (br, 8H), 2.57 (t, J=8.0 Hz, 2H), 2.32 (s, 3H).

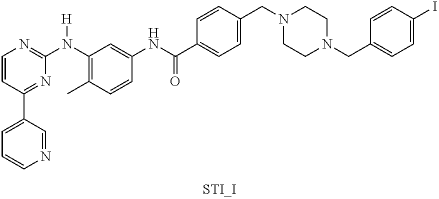

MS: 588.6 (M+H). NMR: ¹H NMR (DMSO) δ 10.16 (S, 1H), 9.29 (d, 1H), 8.698 (S, 1H), 8.67 (dd, J=6.5, 1.5 Hz, 1H), 8.51 (d, J=8.0 Hz, 1H), 8.48 (d, J=5 Hz, 1H), 8.07 (s, 1H), 7.89 (d, J=8.5 Hz, 2H), 7.66 (dd, J=8.0, 1.5 Hz, 2H), 7.52 (dd, J=8, 1.5 Hz, 1H), 7.47 (d, J=5 Hz, 1H), 7.43 (m, 3H), 7.20 (d, J=8.5.0 Hz, 1H), 7.10 (d, J=8.0 Hz, 2H), 3.53 (s, 2H), 2.24 (s, 2H), 2.70 (br, 8H), 2.22 (s, 3H).

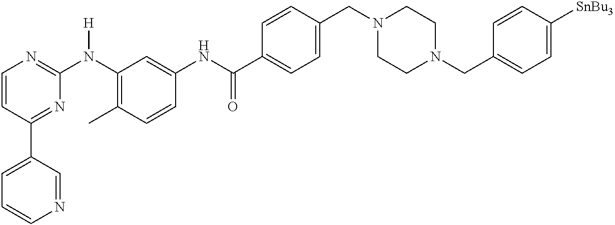

MS: 860.0 (M+H). NMR: ¹H NMR (DMSO) δ 10.16 (S, 1H), 9.29 (d, 1H), 8.97 (S, 1H), 8.67 (dd, J=6.5, 1.5 Hz, 1H), 8.58 (d, 1H), 8.51 (d, J=8.0 Hz, 1H), 8.48 (d, J=5 Hz, 3H), 8.10 (s, 1H), 7.89 (d, J=8.5 Hz, 2H), 7.45 (m, 6H), 7.22 (d, 2H), 3.53 (s, 2H), 3.42 (s, 2H), 2.70 (br, 8H), 2.22 (s, 3H), 1.48 (m, 6H), 1.27 (m, 6H), 0.86 (t, 6H), 0.81 (t, 9H).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Berman, H. M., Westbrook, J., Feng, Z., Gilliland, G., Bhat, T. N., Weissig, H., Shindyalov, I. N., Bourne, P. E. (2000) The Protein Data Bank Nucleic Acids Res 28, 235-42.
Sybyl 7.2, Tripos, Inc., 1699 South Hanley Road, St. Louis, Mo., 63144, USA
Kramer, B., Rarey, M., Lengauer, T. (1999) Evaluation of the FLEXX incremental construction algorithm for protein-ligand docking Proteins 37, 228-41.
Rarey, M., Wefing, S., Lengauer, T. (1996) Placement of medium-sized molecular fragments into active sites of proteins J Comput Aided Mol Des 10, 41-54.
Balzarini, J., Liekens, S., Solaroli, N., El Omari, K., Stammers, D. K., Karlsson, A. (2006) Engineering of a Single Conserved Amino Acid Residue of Herpes Simplex Virus Type 1 Thymidine Kinase Allows a Predominant Shift from Pyrimidine to Purine Nucleoside Phosphorylation J Biol Chem 281, 19273-19279.
Tanis, S. P., Parker, T. T., Colca, J. R., Fisher, R. M. & Kletzein, R. F. Synthesis and biological activity of metabolites of the antidiabetic, antihyperglycemic agent pioglitazone. Journal of Medicinal Chemistry 39, 5053-5063 (1996).
Kompella, A., Bhujanga, R., Adibhatla & Kali, S. in WO2004108699A1 (2004).
Kim, D.-Y. et al. (US20040248918A1, 2004).
Lombardino, J. G. & Niantic, C. (U.S. Pat. No. 4,623,486, 1985).
Alauddin M M, Conti P S, Fissekis J D. J. Labelled. Comp. Radiopharm. 2002; 45: 583-590.
Alauddin M M, Conti P S, and Fissekis J D. J. Labelled. Comp. Radiopharm. 2003; 46: 285-289.

What is claimed is:

1. A method of synthesizing a radiohaloimatinib or a salt thereof, comprising:
condensing a radiohalopiperazine benzoic acid chloride with N-(2-Methyl-5-aminophenyl)-4-(3-pyridyl)-2-pyrimidine-amine, to yield the radiohaloimatinib or a salt thereof.

2. A method of positron emission tomography (PET) imaging of cancer cells in a mammal to detect the levels of expression or activity of a kinase by the cancer cells, comprising:
administering to the mammal a composition containing a radiohaloimatinib or a salt thereof, and
imaging the mammal with PET.

3. The method of claim 2, wherein the radiohaloimatinib is selected from the group consisting of 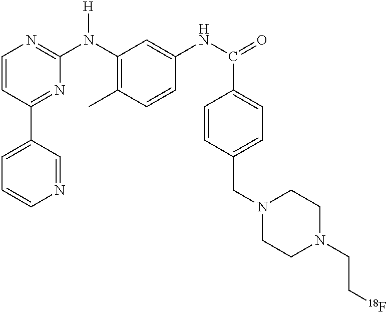
and salts thereof.

4. The method of claim 2, wherein the cancer cells are selected from the group consisting of seminoma cells, acute myelogenous leukemia (AML) cells, and gastrointestinal stromal tumor (GIST) cells.
